(19) United States
(12) Patent Application Publication
(10) Pub. No.: US 2026/0237127 A1
(43) Pub. Date: Aug. 13, 2026

(54) PROJECTION-BASED MEDICAL IMAGE RECONSTRUCTION SYSTEM

(71) Applicant: GACHON UNIVERSITY OF INDUSTRY-ACADEMIC COOPERATION FOUNDATION, Seongnam-si, Gyeonggi-do (KR)

(72) Inventors: Young-Don SON, Incheon (KR); Tae-Jin KIM, Incheon (KR); Hang-Keun KIM, Incheon (KR); Chang-Ki KANG, Incheon (KR); Jong-Hoon KIM, Seoul (KR)

(73) Assignee: GACHON UNIVERSITY OF INDUSTRY-ACADEMIC COOPERATION FOUNDATION, Seongnam-si, Gyeonggi-do (KR)

(21) Appl. No.: 18/701,210

(22) PCT Filed: Oct. 12, 2022
(86) PCT No.: PCT/KR2022/015391

§ 371 Date: Jul. 14, 2025

(30) Foreign Application Priority Data
Oct. 15, 2021 (KR) 10-2021-0137029

(51) Int. Cl. G06T 12/10; G06N 3/0464; G06N 3/0985; G06T 11/00

(52) U.S. Cl. CPC G06T 12/10; G06N 3/0464; G06N 3/0985; G06T 11/00; G06T 2210/41

(57) ABSTRACT

Disclosed in the present technology is a projection-based medical image reconstruction system. According to a specific embodiment of the present technology, comprised are: a sinogram pre-processing unit which pre-processes a sinogram generated with respect to an obtained image and outputs an input sinogram of an image reconstruction neural network, wherein the image reconstruction neural network is provided to reconstruct data of the input sinogram into an image; a pre-filtering module which filters the input sinogram with a weight derived by training a learning model construction unit and outputs the filtered sinogram; a domain transformation module which transforms the filtered sinogram into an image domain by training the learning model construction unit; and a post-filtering module which filters the transformed image with the weight derived by training the learning model construction unit and outputs a reconstructed image. Accordingly, after optimized filtering is performed on a preprocessed input sinogram by updating a weight of an operation with a value of a loss function according to learning results, domain transformation is performed, such that performance and quality of image reconstruction may be improved through artificial-neural-network-based learning without complicated modeling or parameter settings.

### TECHNICAL FIELD

The present disclosure relates to a projection-based medical image reconstruction system and, more specifically, to a technology that improves the performance of a system and the quality of reconstructed images by performing data-based high-frequency filtering on an input sinogram and then doing a domain transform on a filtered sinogram.

### BACKGROUND ART

Projection-based medical image systems such as computed tomography (CT), positron emission tomography (PET), and single photon emission computed tomography (SPECT) obtain sinogram data on which Radon transform is performed with respect to a subject to be imaged by obtaining radiation-based projection data. Algorithms for reconstructing the data obtained in the way into medical images are largely divided into two types.

The first image reconstruction is an analytic reconstruction method, and a filtered back projection (FBP) is a representative example. FBP applies an appropriate high-pass filter such as a one-dimensional ramp filter to each angular view data of the obtained sinogram data and then performs a back-projection according to a corresponding angle. The back-projected images for all angles are accumulated in a designated pixels to reconstruct an image. However, the image reconstructed using FBP reaches a limitation in that the quality is easily degraded by noise included in the data.

The second image reconstruction is an iterative image reconstruction method such as maximum likelihood expectation maximization (MLEM) and ordered subsets expectation maximization (OSEM).

This iterative image reconstruction obtain the estimated sinogram data by performing a projection after estimating an initial image. A correction value was calculated by comparing the actually measured sinogram data with the estimated sinogram data. The estimated image was updated by performing a back-projection with the correction value. This process is iteratively repeated until a convergence condition is reached.

At this time, the iterative image reconstruction has an advantage in that it is easy to improve the quality of an image by applying the physical processes of radioactive particles through statistical modeling in the projection and back-projection processes.

As a result, the iterative reconstruction method that reflects the physical processes is less noisy and reconstructs an image more accurately than the FBP described above, but it takes a long time to process due to the iterative process and has a limitation that it is complex setting convergence conditions for updating the estimated image and stopping the iteration.

Accordingly, the present applicant proposes an image reconstruction method that performs high-frequency filtering with a weight derived from learning on input sinogram data which are based on data of sensor domains (hereinafter abbreviated as sinogram) and then performs a domain transform.

### RELATED ART TECHNICAL LITERATURE

### Non-Patent Literature

Non-patent literature 1: D P Kingma and J Ba, “Adam: A Method for Stochastic Optimization,” ICLR 2015.

### DISCLOSURE

### Technical Problem

Therefore, the present disclosure is to provide a projection-based medical image reconstruction system that improves the performance of a medical image reconstruction system and the quality of reconstructed images by performing high-frequency filtering by applying to an input sinogram an optimized high-frequency filter with a weight derived through learning and then by performing a domain transform.

The objective of the present disclosure is not limited to the objective mentioned above, and other objectives and advantages of the present disclosure that are not mentioned may be understood by the following description, and will be more clearly understood by the exemplary embodiments of the present disclosure. In addition, it will be readily apparent that the objectives and advantages of the present disclosure may be realized by means and combinations thereof indicated in the claims.

The projection-based medical image reconstruction system according to an exemplary embodiment of the present disclosure may
include a sinogram pre-processing unit that pre-processes a sinogram generated for an image to be used for training to output an input sinogram to be input to a neural network for image reconstruction, and
the image reconstruction neural network,
that reconstructs images from input sinograms, includes
a pre-filtering module that performs an operation on the input sinogram with optimal weights derived through training of a learning model construction unit to output filtered sinograms,
a domain transform module that transforms the filtered sinogram into an image domain through training of the learning model construction unit, and
a post-filtering module that performs an operation on the transformed image with a weight derived through training of the learning model construction unit to output a reconstructed image.

Preferably, the learning model construction unit may be provided
to store a training data, which is a pair of the input sinogram and an normalized image having a field of view (FOV) to be used for training and,
to calculate a weight based on a difference between the reconstructed image and the normalized image using a predefined loss function, and
to sequentially update weights of the post-filtering module, the domain transform module using the calculated weight.

Preferably, the pre-filtering module may be provided to have at least one convolution layer in a stacked structure and to sequentially perform a convolution operation on the input sinogram.

Preferably, the domain transform module may be provided
to have at least one fully connected layer in a stacked structure and to sequentially perform an operation on the input sinogram.

Preferably, the post-filtering module may be provided
to have at least one convolution layer in a stacked structure and to sequentially perform a convolution operation on the input sinogram.

Preferably, the sinogram pre-processing unit may be provided
to perform a padding by duplicating an angular view of the sinogram with respect to the field of view (FOV) image and to pad radial bins outside the FOV with ‘0’.

Preferably, the sinogram pre-processing unit may be provided
to pad the upper end of the FOV by duplicating the lower sinogram of the FOV as much as a predetermined size of the area to be padded among angle views of the sinogram with respect to the field Of view (FOV) image and then by flipping left and right and up and down, and
to pad the lower end of the FOV by duplicating the upper sinogram of the FOV as much as a predetermined size of the area to be padded and then by flipping left and right and up and down.

### Advantageous Effects

According to an exemplary embodiment, the performance and quality of image reconstruction may be improved through an artificial neural network-based learning without complex modeling or parameter settings by performing optimized high-frequency filtering and then performing the domain transform on the input sinogram according to updating the weights of the convolution operation of the image reconstruction neural network with weights as a result of learning.

### DESCRIPTION OF DRAWINGS

The following drawings attached in the present specification may illustrate preferred exemplary embodiments of the present disclosure, and serve to help further understand the technical idea of the present disclosure together with detailed descriptions of the present disclosure to be described below, and the present disclosure should not be construed as limited to what is shown in such drawings.

FIG. 1 is a view showing a configuration diagram of an image reconstruction system to which an exemplary embodiment is applied.

FIG. 2 is a view showing a detailed configuration diagram of a sinogram generation unit of FIG. 1.

FIG. 3 is an exemplary view showing a padded sinogram of a FOV of a sinogram of FIG. 1.

FIG. 4 is an exemplary view showing a pre-processed image of a generated sinogram of FIG. 1.

FIG. 5 is a view showing a detailed configuration diagram of an image reconstruction neural network of FIG. 1.

FIG. 6 is an exemplary view showing a visualization of frequency characteristics of a pre-filtering module of FIG. 1.

FIG. 7 is an exemplary view showing a reconstructed image of an input image for each learning model trained by adding a blurring and Poisson noise to data in an exemplary embodiment.

### BEST MODE

Specific structural or functional descriptions with respect to exemplary embodiments are disclosed merely for illustrative purposes, and may be changed and practiced in a variety of forms. Accordingly, exemplary embodiments are not limited to specific disclosure forms, and the scope of the present specification includes changes, equivalents, or substitutes included in the technical idea.

Terms such as “a first” or “a second” may be used to describe a variety of components, but such terms should be interpreted only for the purpose of distinguishing one component from another. For example, a first component may be referred to as a second component, and similarly a second component may also be referred to as a first component.

When a component is referred to as being “connected” to another component, it should be understood that it may be directly connected or connected to the other component, but that another component may exist in between.

Singular expressions include plural expressions unless the context clearly indicates otherwise. In the present specification, the term such as “include” or “have” is intended to designate the presence of the described feature, number, step, action, component, part or combination thereof, and it should be understood not to preclude the presence or additional possibility of one or more other features numbers, steps, actions, components, parts or combinations thereof.

Unless otherwise defined, all terms used herein, including technical or scientific terms, have the same meaning as those generally understood by those skilled in the art. Terms such as those defined in commonly used dictionaries should be construed as having a meaning consistent with the contextual meaning of the relevant technology and are not construed as ideal or excessively formal unless explicitly defined herein.

Hereinafter, preferred exemplary embodiments of the present disclosure will be described in detail with reference to the accompanying drawings.

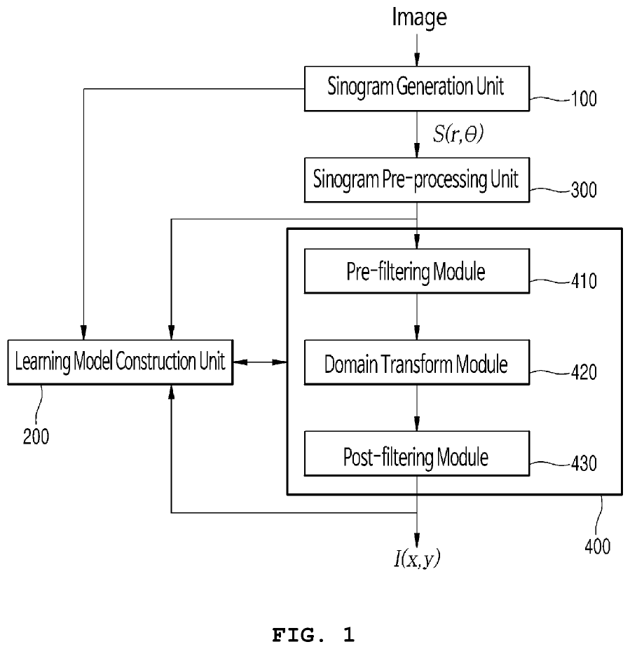

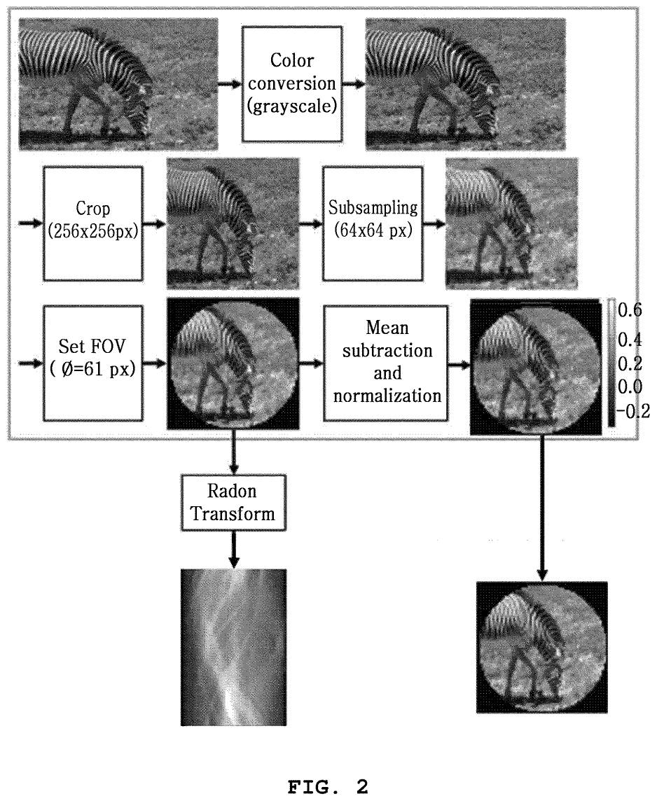

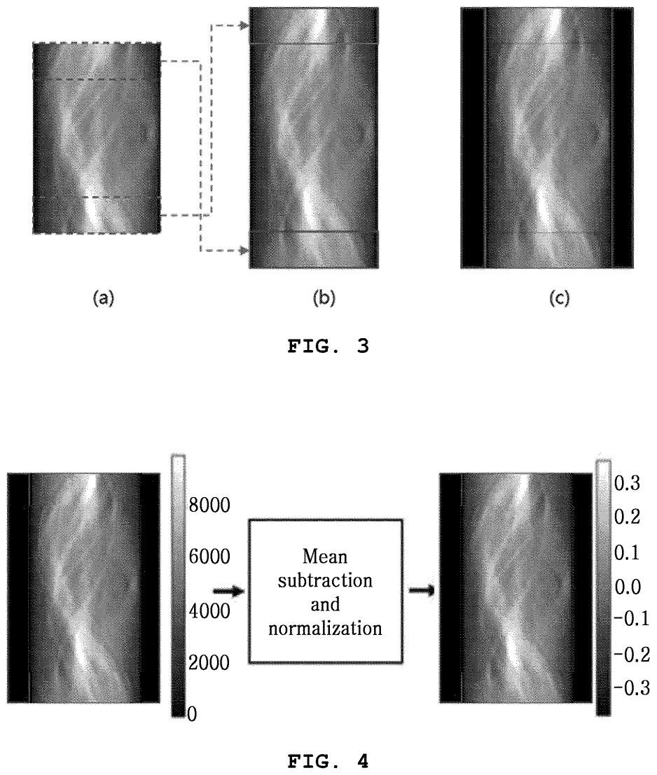

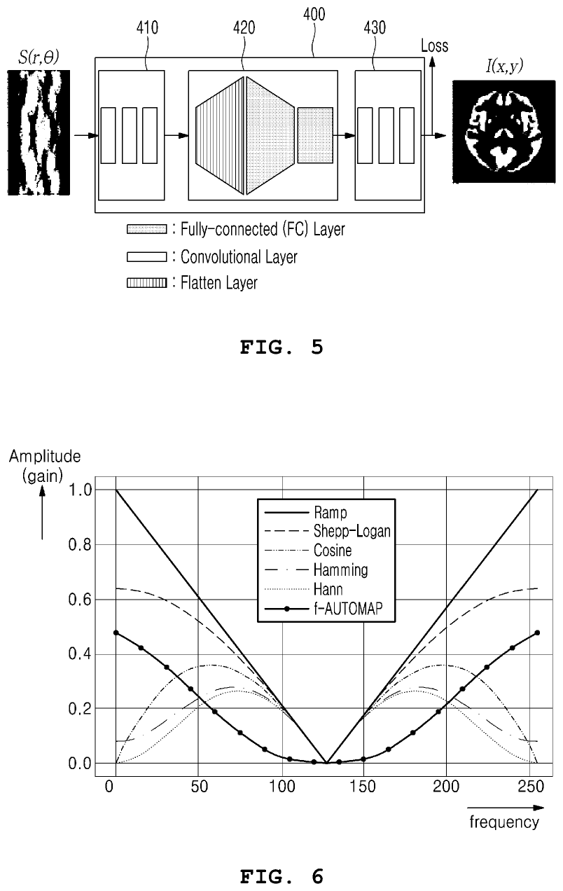

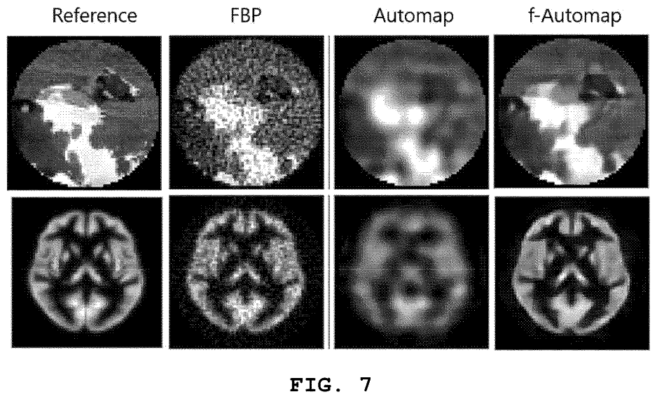

FIG. 1 is a view showing a configuration diagram of an image reconstruction system to which an exemplary embodiment is applied, FIG. 2 is an exemplary view showing a configuration of a sinogram generation unit of FIG. 1, FIG. 3 is an exemplary view showing a sinogram padded in a sinogram pre-processing unit of FIG. 1, FIG. 4 is an exemplary view showing a pre-processed sinogram on the basis of a sinogram pre-processing unit of FIG. 1, FIG. 5 is a view showing a detailed configuration diagram of an image reconstruction neural network of FIG. 1, FIG. 6 is an exemplary view showing a visualization of frequency characteristics of a pre-filtering module of FIG. 1, and FIG. 7 is an exemplary view showing a reconstructed image of an input image for each learning model trained by adding a blurring and Poisson noise to data in an exemplary embodiment.

Referring to FIGS. 1 to 7, a projection-based medical image reconstruction system according to an exemplary embodiment may be provided to obtain a reconstructed image by performing an optimized high-frequency filtering on an input sinogram according to updating a weight of a convolution operation with a weight obtained through learning and then by transforming into an image domain through learning on the filtered sinogram and may include a sinogram generation unit 100, a learning model construction unit 200, a sinogram pre-processing unit 300, and an image reconstruction neural network 400.

As shown in FIG. 2, the sinogram generation unit 100 may apply in the training process a process of generating a sinogram by pre-processing a variety of input images and performing a Radon transform on the training data pairs and may apply in actual medical imaging systems a process of obtaining a sinogram through hardware including a detector of a medical imaging system. Although a process of constructing a learning model by pre-processing and generating training data with respect to a variety of input images is not specifically described in the present specification, it should be understood at the level of those skilled in the art.

For example, the sinogram generation unit 100 may transform an input image to be used for training into a sinogram, and a process of transforming into a sinogram a medical image input to be used for training is as follows.

That is, the sinogram generation unit 100 may change into grayscale the medical image inputted from the open data set which is selected for training and then crop the image as much as a size of 256×256 pixels from the center of the image. After subsampling the image to a size of 64×64 pixels according to the size of the target output image, the value of the pixel outside the FOV may be changed into ‘1’ by applying a circular field of view (FOV) having a diameter of 61 pixels.

The sinogram generation unit 100 may generate a normalized image by subtracting each average value from the circular field of view (FOV) image, and dividing by a maximum value of a determined total data to normalize and pre-process, and may transmit the generated normalized image to the learning model construction unit 200.

Meanwhile, the sinogram generation unit 100 may transform into a sinogram S(r,θ) an image in which a circular field of view (FOV) is processed until a step before normalization by applying the Radon transform and the transformed sinogram S(r,θ) may be transmitted to the sinogram pre-processing unit 300.

The sinogram pre-processing unit 300 may output an input sinogram of the image reconstruction neural network 400 through the padding and normalization with respect to the transformed sinogram.

As an example, as shown in FIG. 3, the sinogram pre-processing unit 300 may perform a padding on the up, down, left, and right sides of the transformed sinogram according to a kernel size of a convolution in order not to change the size of the transformed sinogram (shown in FIG. 3(a)) when performing the convolution. At this time, the padding on the upper and lower portions of the input sinogram may be done by duplicating the lower sinogram as much as a predetermined size and then flipping left and right and up and down to pad the upper sinogram, and likewise by duplicating the upper sinogram as much as a predetermined size and then flipping left and right and up and down to pad the lower sinogram (shown in FIG. 3(b)).

In addition, the sinogram pre-processing unit 200 may perform the padding in a way that duplicates the surrounding angle views, and the left and right padding of the input sinogram may be padded with ‘0’ as much as a predetermined padding size (shown in FIG. 3(c)).

As shown in FIG. 4, the sinogram pre-processing unit 300 may pre-process the padded sinogram through the normalization and then generate an input sinogram in the form of data.

Accordingly, the sinogram pre-processing unit 300 may perform the pre-processing and padding of the above-described sinogram and output an input sinogram of the image reconstruction neural network 400. In addition, the input sinogram of the sinogram pre-processing unit 300 may be transmitted to the learning model construction unit 200.

Accordingly, the learning model construction unit 200 may generate a learning data pair to be used for training with the normalized image obtained from the sinogram generation unit 200 and the input sinogram obtained from the sinogram pre-processing unit 200, and may construct a learning model with the generated learning data pair. In this case, the learning data may be separated into each of 117,465, 4,958, and 40,410 pairs with respect to a total of 162,833 pairs of sinogram and normalized image data and may be used as training, validation, and test data sets.

As shown in FIG. 5, the image reconstruction neural network 400 of the image reconstruction system according to an exemplary embodiment of the present disclosure may include a pre-filtering module 410, a pre-filtering module 420, and a post-filtering module 430, and as an example, the one image reconstruction neural network 400 and the learning model construction unit 100 may be both provided in one device and directly connected through a wire or a connector.

In another example as shown in FIG. 5, the image reconstruction neural network 400 and the learning model construction unit 100 may be configured separately, and may be formed in a structure connected through wireless communication or wired communication between these separated components, but is not limited thereto.

The pre-filtering module 410 may be configured in a stacked structure of at least one (preferably three) convolution layer and perform a filtering on the input sinogram. For example, the first and second convolution layers of the pre-filtering module 410 may comprise 64 3×3 kernels in stride 1, and the third convolution layer may comprise one 1×1 kernel in stride 1.

The weight of each convolution layer of the pre-filtering module 410 may be updated with a weight received through learning of the learning model construction unit 100. That is, an optimized filter may be at least one convolution layer which is updated with the received weight of the learning model construction unit 100.

Herein, the weight received through learning of the learning model construction unit 200 may be derived on the basis of the derived difference between the reconstructed image I(X, Y) and the normalized image by deriving a difference between the reconstructed image I(X, Y) and the normalized image on the basis of a predefined loss function, and described will be a series of processes of deriving on the basis of the derived difference between the reconstructed image I(X, Y) and the normalized image by deriving an difference between the reconstructed image I(X, Y) and the normalized image on the basis of a predefined loss function in the learning model construction unit 200 in the present specification.

Referring to FIG. 6, it may be seen that the convolution layer of the pre-filtering module 310 is a data-based high-frequency filter when comparing the gain of the optimized high-frequency filter based on the convolution layer where the weight is updated through learning and the frequency characteristics of representative high-frequency filters such as Ramp filter, Shepp-Logan filter, Cosine filter, Hamming filter, Hann filter, and the like.

The pre-filtering module 410 may perform the high-frequency filtering through the convolution layer of the batch size 32, and then the filtered sinogram may be transmitted to the domain transform module 320.

The domain transform module 420 may be provided as an artificial neural network structure of a fully connected layer in which an input node and an output node are connected, and two fully connected layers to which the hyperbolic tangent is applied as an activation function may perform the image domain transform on the filtered sinogram of the pre-filtering module 310 through learning.

In the domain transform module 400 of the fully connected layer, the process in which the hyperbolic tangent performs the domain transform using the activation function and converts into a domain-transformed image domain may be the same as or similar to the process of projecting back with each bin value of the sinogram and each voxel value of the reconstructed image.

Then, the image of the domain transform module 420 may be transmitted to the post-filtering module 430, and the post-filtering module 430 may output a reconstructed image (I(x, y)) by removing noise or blurring components included in the image.

Meanwhile, the increment of the weight which is calculated according to the difference between the derived estimated image and the correct answer after deriving a difference between the reconstructed image I(X, Y) and the normalized image on the basis of the loss function predefined in the learning model construction unit 200 may be sequentially transmitted and applied to each weight of the post-filtering module 430, the domain transform module 420, and the pre-filtering module 410 of the image reconstruction neural network 400.

Meanwhile, the learning model construction unit 200 may learn to find an optimized weight parameter through the tuning of hyperparameters such as the number of convolution layers (the number of pre-filter layers), the kernel dimension, the kernel size, the receptive field, and the like.

The hyperparameters here are shown in Table 1 below.

TABLE 1

| The number of | Kernel | Kernel size | |
|---|---|---|---|
| per-filter layers | dimension | (row, column) | Receptive field |
| 1 | 1D | 1, 11 | 1, 11 |
| 1 | 2D | 3, 3 | 3, 3 |
| 1 | 2D | 5, 5 | 5, 5 |
| 3 | 2D | 3, 3 | 7, 7 |

As an example, a difference between the reconstructed image I(X, Y) of the pixel-wise and the normalized image may be derived on the basis of the loss function defined by the weight-sum of L2 loss and gradient loss disclosed in the related art literature 1, and as another example, the difference in the structural similarity including the edge may be derived.

For example, the learning may be performed over a total of 1000 epochs using an ADAM optimizer with a batch size of 32, a learning rate of 104, and a weight attenuation of 0.9.

Accordingly, the learning model construction unit 200 may derive weights on the basis of the difference between the reconstructed images I(X, Y) and the normalized image, and the derived weights may be sequentially transmitted to the post-filtering module 430, the domain transform module 420, and the pre-filtering module 410.

Meanwhile, the image reconstruction neural network 400 may derive a normalized root mean square error (NRMSE) or a structural similarity index measure (SSIM) on the basis of the learning result and may perform a learning evaluation on the basis of the derived NRMSE or SSIM.

Herein, NRMSE may be a pixel-unit difference between the reconstructed image and the normalized image and may be defined as in Equation 1 below.

$$M S E = \frac{1}{n} {\sum }_{i = 1}^{n} {( {y}_{i} - {\hat{y}}_{i} )}^{2} N R M S E = \frac{1}{\overline{y}} \sqrt{M S E} \qquad [ Equation l ]$$

NRMSE may be an index where RMSE (root mean squared error) is divided by the average value y of the normalized image, and indicates that as NRMSE is closer to 0, the reconstructed image and the normalized image are more matched.

In addition, SSIM may indicate the structural similarity between the reconstructed image and the normalized image and may be defined by the following equation 2.

$$S S I M ( a , b ) = \frac{( 2 {μ}_{a} {μ}_{b} + {c}_{1} ) ( 2 {σ}_{ab} + {c}_{2} )}{( {μ}_{a}^{2} + {μ}_{b}^{2} + {c}_{1} ) ( {σ}_{a}^{2} + {σ}_{b}^{2} + {c}_{2} )} \qquad [ Equation 2 ]$$

where a and b are the windows of size M×N used for the two images whose similarity is to be calculated respectively. μ and σ are the average and standard deviation of each window, and c₁ and c₂ are variables for adjusting the indicator values. SSIM is a value between 0 and 1, and a value close to 1 indicates that the two images are structurally similar.

Table 2 below is an exemplary diagram showing NRMSE and SSIM for each learning model, and referring to Table 2, it may be seen that NRMSE is closer to 0 and SSIM is closer to 1 in the image reconstruction performance of the image reconstruction neural network according to an exemplary embodiment compared to the learning model 1 (FBP with a Ramp filter applied), the learning model 2 (AUTOMAP) and the learning model 3 (f-AUTOMAP).

TABLE 2

| | Reconstruction Performance | |
|---|---|---|
| Model | NRMSE ± SD | SSIM ± SD |
| FBP (ramp filter) | 0.108 ± 0.029 | 0.959 ± 0.011 |
| AUTOMAP | 0.050 ± 0.019 | 0.983 ± 0.008 |
| f-AUTOMAP | 0.003 ± 0.002 | 1.000 ± 0.001 |

Accordingly, the image reconstruction neural network may be evaluated to have higher performance and quality of the learning model as the derived NRMSE approaches 0, and be evaluated to have higher performance and quality of the learning model as the derived SSIM approaches 1.

FIG. 7 is an example diagram showing a reconstructed image for an input image with respect to each learning model learned by adding blurring and Poisson noise to the data, and referring to FIG. 7, it may be seen that the performance and quality of the learning model 3 (f-AUTOMAP), whose derived SSIM is closest to 1, is higher compared to the learning models 1 and 2.

Accordingly, the optimized high-frequency filtering in an exemplary embodiment may be performed on the pre-processed input sinogram according to updating the weight of the convolution operation with the weight of the learning result and then the image domain transform may be performed on the filtered sinogram, thereby improving the performance and quality of the image reconstruction system.

Although exemplary embodiments have been described with limited drawings as described above, those skilled in the art may apply various technical modifications and variations based on the above. For example, an appropriate result may be achieved even when the described techniques are performed in a different order from the described method, and/or the components of the described systems, structures, devices, circuits, etc. may be combined or combined in a different form than described, or substituted or replaced by other components or equivalents.

### DESCRIPTION OF REFERENCE NUMERALS

100: sinogram generation unit
200: learning model construction unit
300: sinogram pre-processing unit
400: image reconstruction neural network
410: pre-filtering module
420: domain transform module
430: post-filtering module

### INDUSTRIAL APPLICABILITY

The present disclosure may bring great advances in the accuracy and reliability of operation and furthermore performance efficiency with respect to the projection-based medical image reconstruction system through learning on the basis of an artificial neural network without complex modeling or parameter settings according to performing a domain transform after performing the optimized filtering on the pre-processed input sinogram by updating the weight of the convolution operation with the weight according to the learning result, may be applied to a variety of fields, may actively utilize environmental monitoring in related industries by securing deep learning-based image reconstruction models and core technologies for a nuclear medical image reconstruction, and may have not only a sufficient possibility of commercializing or selling the nuclear medicine image reconstruction system but also industrial applicability as much as it may also be clearly implemented in reality.

1. A projection-based medical image reconstruction system, the system comprising:
a sinogram pre-processing unit that pre-processes a sinogram to output an input sinogram to be input to an image reconstruction neural network; and
the image reconstruction neural network, comprising:
a pre-filtering module that performs an operation on the input sinogram with a weight derived through training of a learning model construction unit to output a filtered sonogram;
a domain transform module that transforms the filtered sinogram into an image domain through training of the learning model construction unit; and
a post-filtering module that performs an operation on the transformed image with the weight derived through training of the learning model construction unit to output a reconstructed image.

2. The system of claim 1, wherein the learning model construction unit is provided to store a training data pair of the input sinogram and an image normalized for the image of a field of view (FOV) to be used for training as a learning model, to calculate a weight based on a difference between the reconstructed image and the normalized image using a predefined loss function, and to sequentially update weights of the post-filtering module, the domain transform module, and the pre-filtering module using the calculated weight.

3. The system of claim 1, wherein the pre-filtering module is provided to have at least one convolution layer in a stacked structure and sequentially perform a convolution operation on the input sinogram.

4. The system of claim 1, wherein the domain transform module is provided to have at least one fully connected layer in a stacked structure and sequentially perform the operation on the input sinogram.

5. The system of claim 1, wherein the post-filtering module is provided to have at least one convolution layer in a stacked structure and sequentially perform a convolution operation on the input sinogram.

6. The system of claim 1, wherein the sinogram pre-processing unit performs a padding by duplicating an angle view of the sinogram for a field of view (FOV) image and pads a radial bin outside of the FOV with ‘0’.

7. The system of claim 5, wherein the sinogram pre-processing unit is provided to pad an upper end of a field of view (FOV) by duplicating a lower sinogram of the FOV as much as a predetermined size of an area to be padded among angle views of the sinogram with respect to the FOV image and by flipping left and right and up and down, and to pad a lower end of the FOV by duplicating a upper sinogram of the FOV as much as a predetermined size of the area to be padded and by flipping left and right and up and down.
